(19) United States
(12) Patent Application Publication
(10) Pub. No.: US 2010/0092338 A1
(43) Pub. Date: Apr. 15, 2010

(54) Test element magazine

(76) Inventors: Hans List, Hesseneck-Kailbach (DE); Hans-Peter Haar, Wiesloch (DE); George Bevan Kirby Meacham, Shaker Heights, OH (US)

Correspondence Address: ROCHE DIAGNOSTICS OPERATIONS INC., 9115 Hague Road, Indianapolis, IN, 46250-0457, US

(21) Appl. No.: 12/622,595

(22) Filed: Nov. 20, 2009

Related U.S. Application Data
(63) Continuation of application No. PCT/EP2008/056407, filed on May 26, 2008.
(60) Provisional application No. 60/940,561, filed on May 29, 2007.

(51) Int. Cl. G01N 33/50; A61B 5/151; G01N 33/48; A61B 5/157

(52) U.S. Cl. 422/58; 422/104; 422/100; 204/403.02; 600/583

(57) ABSTRACT

A test element magazine for a system for measuring the concentration of at least one constituent in a fluid sample is provided. The test element magazine comprises at least one test element in at least one chamber. The chamber is individually sealed by a sealing element. The test element comprises at least one active portion for contacting a skin portion of a patient and/or for contacting a fluid sample, wherein the test element further comprises at least one support body, wherein the support body is adapted for being engaged by an actuator. By a forward movement of the test element the support body of the test element applies a force to the sealing element, thereby forcing open the sealing of the chamber.

### CROSS-REFERENCE TO RELATED APPLICATIONS

This application is a continuation of PCT/EP2008/056407, filed May 26, 2008, which is based on and claims priority to U.S. Provisional Application Ser. No. 60/940,561, filed May 29, 2007, which is hereby incorporated by reference.

### BACKGROUND

The present disclosure relates to a test element magazine, which may be used in a system for measuring the concentration of at least one constituent in a fluid sample. The present disclosure further refers to a test system for measuring a concentration of at least one constituent in a fluid sample, the test system comprising at least one test element magazine according to the present disclosure. The test element magazine and the test system according to the present disclosure may specifically be used in the field of biomedical analytical systems, such as in the field of measuring a concentration of an analyte, such as glucose, cholesterol or similar analytes in a body fluid sample, such as blood, urine or other body fluids.

The determination of the concentration of certain constituents (also called analytes), such as glucose or cholesterol, in a body fluid, such as blood or urine, forms an essential part of the daily routine of patients suffering from certain diseases, such as, for example, diabetes. Thus, the concentration of the analyte has to be determined in a fast and reliable way, usually several times per day, in order to take appropriate medical measures.

In order not to unduly restrict the daily routine of the patient, in many cases mobile analytical test systems are used, which are easy to transport and to handle. Several mobile systems are commercially available, which employ different measurement principles. A first measurement principle is to use electrochemical measurements, wherein a blood sample obtained from a patient, e.g., by perforating a portion of the patient's skin using a lancet, is applied to an electrode covered with enzymes and mediators. Test elements for these electrochemical measurement concepts are known, e.g., U.S. Pat. No. 5,286,362. Other known measurement principles are to use optical measurements, which are, e.g., based on color changes of certain chemical substances when they contact the analyte to be detected. Systems for detecting those color reactions and the use of these color reactions for determining the concentration of the analyte are known from several prior art documents, such as, for example, CA 2,050,677.

Instead of using separate processes for obtaining the fluid sample and analyzing the fluid sample, concepts and systems are known which are adapted for collecting (sampling) the body fluid and analyzing the body fluid in one step. These concepts are widely known as “get and measure” (GAM) concepts. For example, WO 2005/084546 A2 discloses a body fluid sampling device comprising a skin piercing element having a collection zone for receiving body fluid, wherein the device further comprises a fluid receiving means spaced apart from the collection zone. The fluid receiving means may have a test zone for performing an analytical reaction. The fluid sample from the collection zone is automatically or manually transported to the fluid receiving means to contact the fluid with the test zone.

Similarly, WO 2007/045412 A1 describes a test element for use as a disposable article for examining a body fluid, comprising a piercing element for piercing a body part, a collection zone configured thereon for body fluid obtained through the puncture and at least one optical waveguide for carrying out an optical measurement in the collection zone. The collection zone is configured by a collecting aperture of the piercing element, which aperture is elongate in the direction of piercing, and in that the optical waveguide is integrated into the piercing element so as not to be displaced and is arranged with its distal end in a proximal measuring zone of the collecting aperture.

Nevertheless, a major challenge for GAM systems, as well as for other systems using analytical test elements or “passive” test elements, such as bare lancets, resides in a clean and hygienic storage of the test elements. From the prior art, several concepts are known for storing test elements; in order to keep the test elements protected from external detrimental influences, such as humidity and/or contaminations. WO 03/088834 A1, for example, discloses a cartridge containing a number of penetrating members. The penetrating members are movable to extend radially outward from the cartridge to penetrate tissue. The cartridge contains a plurality of cavities. Each of the cavities is defined in part by a deflectable portion. The deflectable portion in a first position prevents the penetrating member from exiting the cartridge, and the deflectable portion is movable to a second position to create an opening that allows the lancet to extend outward from the cartridge.

As already shown in WO 03/088834 A1, a considerable effort has to be taken, in order to prepare a test element located inside a test element magazine, specifically inside a sealed chamber of a test element magazine, for subsequent use. Thus, concepts are known, using a separate operation of opening a chamber, such as the concept disclosed in WO 03/088834 A1. These concepts avoid contact between the test element and a sealing element of the chambers, and, thus, avoid damages to the test element, which might occur during contact between the test element and the sealing element. Thus, when using aluminum foil as a sealing element of the chambers, a contact between a sharp cutting edge of a lancet of the test element and the aluminum foil may damage the cutting edge. Further, if polymer films or other types of sealing elements are used, a physical contact between the test element and the sealing element may chemically deteriorate the properties of the test element, such as hydrophilic properties of certain get and measure lancets. Nevertheless, concepts using separate opening mechanisms for opening the sealing of the chambers, in order to prepare the test elements for subsequent use, create a considerable additional mechanical effort and complexity. Thus, in most cases, an additional mechanical element is required, in order to open the sealing element of a chamber, in order to prepare the test element for subsequent use.

A simple alternative would be to use a sealing which is directly penetrated by the test element, such as by a lancet of the test element. A proper choice of the sealing element might reduce physical damages to the test element. Thus, if a polymer film barrier is used, a penetration of this polymer film barrier by the lancet will be unlikely to damage the lancet cutting edge. Nevertheless, this penetration action may affect the lancet motion in an unpredictable way. Further, polymer film barriers provide a less effective moisture barrier than metal foils, such as aluminum foils. Still, a physical contact between the test element and the sealing element is likely to deteriorate the properties of the test element, such as the hydrophilic character of the test elements, which is a crucial property of the test element in many get and measure systems.

### SUMMARY

A test element magazine, as well as a test system, comprising the test element magazine, is disclosed. The test element magazines, as well as the test systems, can be designed to be capable of an operation avoiding a separate opening step for opening the sealing of a chamber, and, can, therefore, allow for the use of rather simple actuators for engaging and actuating the test elements. Thus, the overall costs of the magazine as well as the test system according to the present disclosure may be significantly reduced. Further, since no separate opening mechanism is required, the test systems may be designed having smaller dimensions than prior art systems, which is especially advantageous in portable test systems.

In a first embodiment of the present disclosure, a test element magazine is disclosed. The test element magazine may be used for a system for measuring the concentration of at least one constituent in a fluid sample. Alternatively, the test element magazine may be used for storing lancing needles or for storing test elements adapted for determining the concentration of a constituent of a body fluid. Other applications may be possible. The constituent may be an analyte, such as blood glucose or cholesterol, and the fluid sample may be a body fluid sample, such as blood, urine, saliva or other body fluids. Other types of analytes and/or fluid samples may be possible.

The test element magazine can comprise at least one test element in at least one chamber, wherein the chamber is individually sealed by a sealing element. The test element can comprise at least one active portion and at least one support body. The active portion can be adapted for contacting a skin portion of a patient and/or for contacting a fluid sample. Depending on the type of test element, the active portion may comprise at least one of the following: a lancing portion, a sampling portion, an analytical test portion for determining the concentration of the constituent of the body fluid. The support body can be adapted for being engaged by an actuator, such as an actuator for effecting a forward/lancing movement of the test element or for performing a sampling motion. Thus, the support body may be adapted to be coupled to an actuator, such as a gripper and/or a plunger of a test system. For this purpose, the support body may comprise a flat end face for absorbing a momentum transferred by the actuator, or the support body may comprise any other means for momentum transfer, such as a recess and/or a projection capable of being coupled to an actuator. However, other embodiments may be possible.

The support body may be spaced apart from the active portion, such as by arranging the active portion at a front end of the test element and by arranging the support body at a rear/opposite end of the test element. Further, the materials of the active portion and the support body may differ, in accordance with their functionalities. The support body and the active portion may be designed as separate components of the test element. Further, the support body may have a maximum width substantially perpendicular to an axis of the test element (the axis of the lancing motion) which can exceed the maximum width of the active portion, in order for the support body to force open the sealing element. Thus, the test element and/or the support body may comprise a shoulder, a bead, corrugation, a crimp, a pleat, a fin, a seam or a similar element or a combination of elements having a maximum width according to the above-mentioned condition. Other embodiments may be possible.

The test element may comprise a simple lancet for solely perforating the skin portion, without any further sampling and/or testing means and/or analytical test portion, such as a simple lancing needle without any analytical test portion. Alternatively, the test element may comprise a testing means and/or analytical test portion for detecting a constituent in a fluid sample, without incorporating a lancet. Nevertheless, the test element may be designed to act as a GAM test element (such as the test elements disclosed in WO 2005/084546 A2 or WO 2007/045412 A1) and, thus, may comprise sampling and/or testing means for sampling a body fluid and for measuring the concentration of one or more constituents (also called analytes) in the body fluid. The sampling means may comprise a capillary structure within a lancing needle. The testing means may comprise a test strip comprising one or more chemical substances for detecting the constituent. The testing means may be physically attached to the needle or may be positioned within the system, separated from the needle. An optical waveguide system may be used for optical detection of the constituent. Further, the test strip may function as sampling means, which may be connected to the needle or which may be arranged separately. Other embodiments of the test element than the embodiments described may be possible.

The test element magazine can be constructed in such a way that, by a forward movement of the test element within the chamber, the support body of the test element can apply a force to the sealing element, thereby forcing open the sealing of the chamber. The opening force exerted to the sealing element can be directed substantially perpendicular to the sealing element, or can, at least, comprise a directional component substantially perpendicular to the sealing element.

This opening force forcing open the sealing element may be exerted by different way. Thus, the test element magazine may be used in a test system comprising an actuator engaging the test element inside the chamber and enforcing the forward movement of the test element, e.g., by pushing the test element. The actuator may be adapted for forcing apart at least part of the support body of the test element, thereby enlarging the width of the support body, thereby forcing open the sealing of the chamber. Thus, the test element may comprise a hollow rear end portion, wherein the actuator may comprises a plunger entering the rear end portion and thus enlarging the rear end portion, thereby forcing open the sealing element of the chamber.

Additionally, or alternatively, the chamber may comprise a constricted portion, wherein the chamber can be dimensioned in such a way that, by forcing the support body of the test element into the constricted portion of the chamber, a force can be applied to the sealing element, thereby forcing open the sealing of the chamber. Thus, the constricted portion may be shaped in a way that a clearance within this constricted portion can be narrower than a part of the test element, such as the test element support body, which can be pushed through that constricted portion during a motion of the test element, such as during a lancing or sampling motion.

The constricted portion of the chamber may comprise one or more of the following constriction elements: a ramp, allowing for a sliding motion of the test element; a cam protruding into the chamber; a projection protruding into the chamber; and a shoulder protruding into the chamber. Nevertheless, other types of constrictions may be possible, such as, for example, a conical shape of the chamber, wherein a linear motion of the support body of the test element into narrower parts of the conical chamber can provide an opening force for opening the sealing of the chamber. This opening force can have at least one directional component substantially perpendicular to an axis of the test element, in order to allow for an opening motion opening the sealing of the chamber.

The at least one constriction element may at least partially be formed by a magazine body. The magazine body may be formed by a molded plastic element. Thus, the constriction elements may be formed during the molding process of the magazine body.

The sealing element may further comprise at least one lid portion. This lid portion may at least partially cover the chamber. The sealing element may further comprise at least one sealing support, wherein the lid portion can be connected to the sealing support by a hinge. Thus, the lid portion may provide external access between the test element and the fluid sample, such as a skin portion of a patient, through a “hinged door” opening in the sealing element of the test element magazine, wherein the hinged door openings can be opened by contact between the moving test element and the walls of the chamber. A gap between the sealing support and the lid portion may be covered by a foil or a membrane element. This foil or membrane element may be a frangible element, which may be broken by the door opening motion of the lid portion of the sealing support.

The lid portion may have one or more of the following shapes: an essentially rectangular shape, a lamellar shape, a shape of a piece of pie, or any other suitable shape. Preferably, the geometry of the lid portion and the opening covered by the lid portion can be designed in such a way that the lid motion can allow the test elements to pass through the opening without contact between the test element, e.g., a lancet of the test element, and the openings. The opening covered by the lid portion may be narrower than the test element width, such that the perimeter of the door openings can restrain and guide the test element when the lid portion is in an open position.

The sealing element may comprise at least one foil or membrane element, especially for covering a gap between the sealing support and the lid portion. This foil or membrane element may comprise at least one of the following materials: an aluminum foil, a copper foil, a polyethylene foil, a fluorinated polyethylene foil, a metal-coated polymer foil, such as polyethylene, polyethylene terephtalate (PET) etc. coated with aluminum or other metals or inorganic barrier materials, such as silicon dioxide etc. Other polymer materials and/or metal foil elements can be usable, wherein frangible elements, membranes or foils are used.

The test element magazine may comprise several types of magazines, such as flat or curved magazines comprising one or more test elements. Thus, the test element magazine may comprise an arrangement out of a plurality of possible arrangements of chambers for test elements, wherein the chambers can be arranged in such a way that they may be opened separately. Thus, the test element magazine may comprise a linear arrangement of chambers, a zigzag arrangement, a bent arrangement (such as a belt or strap comprising a plurality of chambers), a circular arrangement, a drum-like arrangement or any other suitable type of arrangement. In the following, without restriction of the scope of the present disclosure, circular arrangements are disclosed. Nevertheless, other types of arrangements may be used.

The test element magazine can comprise a disk magazine, such as a disk magazine having an essentially circular shape. The disk magazine may comprise a plurality of test elements, which can be arranged radially in individual chambers, with the active portions of the test elements pointing outwards. The disk magazine may comprise a central volume of the disk, which can be empty, such as the disk can be free to rotate about a rotating mechanism.

The test element magazine may further comprise a disk body, wherein the sealing element at least partially can cover the disk body. This disk body may comprise one or more molded plastic elements. Thus, the chambers for the test elements may at least partially be formed during a simple molding process.

The test elements may comprise one or more test elements for analyzing the fluid sample, such as, for example, the electrochemical and/or optical test elements known from the prior art. Thus, the test element may comprise a substance that changes at least one chemical or physical property as a function of a concentration of the constituent in the fluid, when bringing the fluid in contact with the substance. The at least one chemical or physical property may comprise an optical property and/or a physical property.

Additionally, or alternatively, to the chemical substance, the test element, specifically the active portion of the test element, may comprise a lancet. A lancet may comprise a needle and/or any other means for perforating or cutting a skin portion of a living organism, such as a human or animal patient.

In one embodiment, the test element may be designed for GAM purposes. Thus, a lancet and a chemical substance may be provided. The lancet may comprise a capillary element for sampling a fluid sample. For this purpose, the lancet may further comprise a hydrophilic surface, preferably the interior surface of the capillary element.

In another embodiment, the support body of the test element may comprise a sealing portion. This sealing portion may form a part of the sealing of the chamber. Thus, the test element may comprise a sterile part, which can be located inside the chamber, and an engagement portion, which is located outside the chamber. The portion in between the engagement portion and the sterile part of the test element may provide a close sealing contact with the chamber walls, thus providing a protection of the sterile part against detrimental influences.

In a further embodiment, a test system for measuring the concentration of at least one constituent (or analyte) in a fluid sample is provided. The test system may comprise a test element magazine. Further, the test system may comprise at least one actuator comprising an engagement portion for engaging a test element of the test element magazine.

The actuator may be adapted for forcing the support body of the test element into the constricted portion of the chamber, thereby forcing open the sealing of the chamber. The actuator may further be adapted for forcing the test element to perform a lancing motion and/or a sample collecting motion.

The engagement portion may comprise an optical port for optically contacting the test element. Thus, optical transmission means may be provided, in order to bring light from a measuring instrument to illuminate a chemical substance undergoing a change in one or more chemical or physical properties as a result of contact with the analyte, as well as optical transmission means to return light to the measuring instrument, in order to measure the color change.

Additionally, or alternatively, the engagement portion may comprise an electrical port for electrically contacting the test element. Thus, the test system may comprise means for electrochemically determining the concentration of the analyte in the body fluid.

Other features of the embodiments of the present disclosure will be apparent in light of the description of the disclosure embodied herein.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1 illustrates a schematic perspective view of a first embodiment of a test element magazine according to an embodiment of the present disclosure.

FIG. 2 illustrates a cross-sectional view of a second embodiment of a test element magazine according to an embodiment of the present disclosure.

FIG. 3 illustrates a top view of a part of the test element magazine of FIG. 2 according to an embodiment of the present disclosure.

FIG. 4 illustrates a cross-section of a third embodiment of a test element magazine according to an embodiment of the present disclosure.

FIG. 5 illustrates a fourth embodiment of a test element magazine according to an embodiment of the present disclosure.

FIG. 6 illustrates an embodiment of a test element according to an embodiment of the present disclosure.

FIG. 7 illustrates a schematic view of an embodiment of a test system according to an embodiment of the present disclosure.

### DETAILED DESCRIPTION

In the following detailed description of the embodiments, reference is made to the accompanying drawings that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

Referring initially to FIG. 1, a perspective view of a first embodiment of a test element magazine 110 is schematically shown. The test element magazine in this embodiment can be a disk magazine of substantially circular shape, with a central opening 112 of circular shape. This central opening 112 may be used for at least partially accommodating a rotating mechanism for an angular positioning the test element magazine 110, and/or may be used for accommodating at least part of an actuator for engaging a test element of the test element magazine 110.

The test element magazine 110 can comprise a number of individually sealed chambers 114, each chamber 114 comprising a test element (not shown in FIG. 1). The test element magazine 110 can comprise a magazine body 116, which may be formed of a molded hard plastic element.

The magazine body 116 can be covered by a sealing element 118, which, in this embodiment, can comprise a dome-shaped molded sealing support 120, which can act as a frame for the sealing element 118 and which can essentially cover the magazine body 116.

The scaling element 118 can further comprise a number of lid portions 122, each lid portion 122 covering one of the chambers 114. Without limitation of the scope of the disclosure, only three of the lid portions 122 and only some of the chambers 114 are depicted in FIG. 1.

The lid portions 122 may also be formed of a hard plastic molded element and may be formed in a single piece with the sealing support 120. The lid portions 122 can be connected to the sealing support 120 by hinges 124, allowing for the lid portions 122 to be opened, i.e., to be deflected from the openings 126 covered by the lid portions, thus clearing these openings 126 for a use of the test element housed in the respective chamber 114. The hinges 124 may simply comprise a connection line between the lid portions 122 and the sealing support 120, or the hinges 124 may comprise a thin, flexible section in the molded plastic part forming the sealing support 120 and the lid portions 122.

In the openings 126, between the lid portions 122 and the sealing support 120, gaps 128 can be formed. In order to close these gaps 128 and to protect the interior of the chambers 114 from moisture and/or contaminations which might ingress through the gaps 128, the sealing element 118 may further comprise a membrane element 130, which at least can partially cover the dome of the sealing support 120. This membrane element 130 may also have a dome-like shape and may be formed of a metal foil, such as an aluminum foil, and, in one exemplary embodiment, can completely cover the gaps 128. Only part of the membrane element 130 is shown in FIG. 1. The membrane element 130 can comprise a frangible membrane element, which may be broken along the line of the gaps 128, as soon as the lid portion 122 of a chamber 114 is opened by pivoting the lid portion 122 around the respective hinge 124. The membrane element 130 may be glued to the sealing support 120, and the sealing support 120 may be glued to the magazine body 116. Other connecting techniques may be used known in the art, such as, for example, welding, crimping, molding or similar techniques.

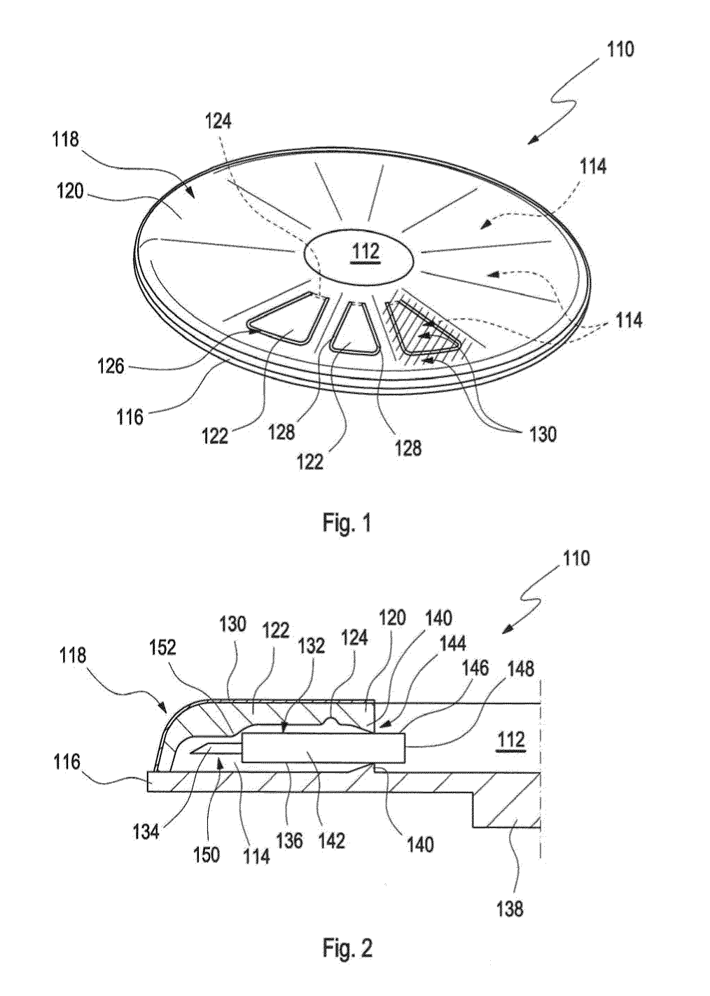

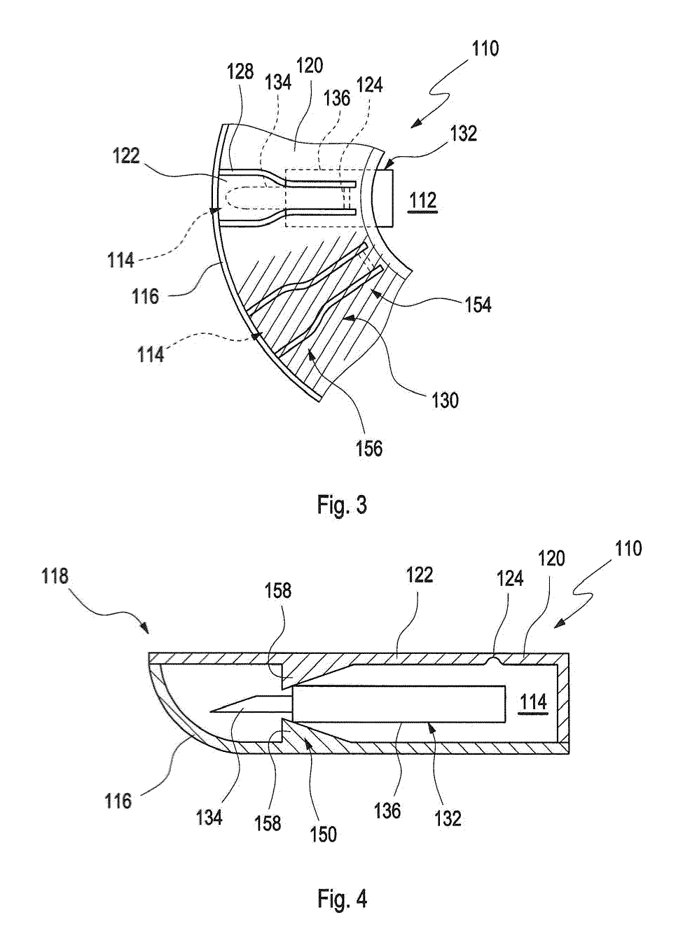

In FIGS. 2 and 3, a second embodiment of a test element magazine 110 is depicted. Therein, FIG. 2 illustrates a sectional side view of a chamber 114 of the test element magazine, comprising a test element 132. Again, as in the exemplary embodiment shown in FIG. 1, the test element magazine 110 can be an essentially circular shape and can comprise a number of radial chambers 114 comprising test elements 132 with a lancet 134 pointing radially outwards. The test elements 132 can further comprise a support body 136, wherein the support body 136 can exhibit a larger cross-section than the lancet 134. Thus, the support body 136 may be formed of a molded plastic material holding the lancet 134.

As can be seen in FIG. 2, similarly to the embodiment in FIG. 1, the test element magazine 110 can comprise a molded magazine body 116, which, in this exemplary embodiment, can be substantially disk-shaped, with a central axial lug 138 and a central circular opening 112, which, in this embodiment, can be formed as a blind hole instead of a through hole. Other suitable embodiments of the central opening 112 are possible.

As in FIG. 1, the magazine body 16 in the embodiment in FIG. 2 can be covered by a sealing element 118 comprising a sealing support 120 (only partially visible in FIG. 2) and a number of lid portions 122. The lid portions 122 and the sealing support 120 may be formed of a molded hard plastic material, preferably in single piece, similarly to the embodiment in FIG. 1. The lid portions 122 and the sealing support 120 can be connected by hinges 124, which can be formed by thin sections in the molded plastic part forming the sealing support 120 and the lid portions 122. In general, the sealing support 120 and the lid portions 122 can form a dome-shaped part, which can essentially cover the magazine body 116 and which may be connected to the magazine body 116 by gluing or other connecting techniques, as described above.

Further, the sealing element 118 can comprise a frangible membrane element 130, which, again, may comprise an aluminum foil, a copper foil, a polymer foil or other frangible elements, and which can cover the sealing support 120 and the lid portions 122, thereby sealing the gaps 128 around the lid portions 122.

As can be seen in FIG. 2, the test elements 132 can be arranged within the chambers 114 in a radial manner. Each test element 132 can partially protrude into the central opening 112 with a rear part of the support body 136. The magazine body 116 and the sealing support 120 can each comprise taper plugs 140 protruding into the chambers 114, wherein the taper plugs 140 can be designed to firmly hold the support body 136 of the test element 132, thereby sealing the chamber 114 from the surrounding, especially from the central opening 114. Thus, each test element 132 virtually can be subdivided into a sterile part 142 arranged inside the sealed chambers 114, a sealing portion 144 cooperating with the taper plugs 140 to seal the chambers 114, and an engagement portion 146 situated outside the chamber 114, protruding into the central opening 112.

The engagement portion 146 may comprise an optical port and/or an electrical port for optically and/or electrically contacting the test element 132. Thus, the optical and/or electrical port 148 may comprise one or more optical fibers, which may be optically contacted, and/or one or more electrical contacts, in order to electrically contact the test element 132. Further, the engagement portion 146 may be used for mechanically engaging the test elements 132, e.g., for projecting the test elements 132 to perform a lancing motion and/or for retracting the test elements 132 after performing the lancing motion, e.g., for collecting a body fluid sample. For this purpose, as will be described in further detail below, an actuator may engage the engagement portion 146 of the test element 132 in a measurement position through the central opening 112 of the test element magazine 110.

Especially in cases where the test elements 132 are formed as get and measure (GAM) test elements, these test elements 132 may comprise an active portion, which can be part of the sterile part 142. In the embodiment shown in FIGS. 2 and 3, the active portion may comprise the lancets 134 of the test elements 132. In many cases, it may be important for these active portions, e.g., the lancets 134, not to touch any part of the magazine body 116 and/or the sealing element 118. Any contact may deteriorate the active portion, e.g., by deteriorating hydrophilic properties of the lancet 134, which again may deteriorate or even prevent the sampling action.

For this purpose, the chambers 114 can comprise constricted portions 150. These constricted portions 150 can be dimensioned in a way that, when pushing the support bodies 136 of the test elements 132 into these constricted portions 150, an opening force can be exerted onto the sealing element 118, thus opening the chambers 114 without the active portions of the test elements 132 touching the magazine body 116 and/or the sealing element 118.

In the embodiment shown in FIGS. 2 and 3, the constricted portion 150 of the chambers 114 can comprise a door opening cam 152. These door opening cams 152 can be part of the lid portions 122 of the sealing element 118 and can protrude into the interior of the chambers 114. By radially pushing outward the test element 132 in FIG. 2 (i.e., exerting a force from the right to the left in FIG. 2), the enlarged support body 136 of the test element 132 can be pressed into the constricted portion 150, can contact the door opening cam 152, and, thus, can open the lid portions 122 by rotating them around the hinges 124, thereby breaking open the frangible membrane element 130 and opening the chamber 114 without the lancet 134 touching the lid portion 122 or any other part of the sealing element 118. Thus, in one single motion, the test element 132 may be used to open the chamber 114 and to perform a lancing motion. No separate mechanism for opening the chambers 114 may be required. The beginning of the outward radial lancet motion can cause the support body 136 of the test element 132 to contact and can lift the cam surfaces of the door opening cams 152 inside the lid portions 122.

In the embodiment in FIG. 1, the lid portions 122 of the sealing element 118 can be more or less shaped like a piece of pie. Nevertheless, the openings 126 covered by the lid portions 122 can preferably be narrow, except where the test elements 132 emerge, so that the test elements can be contained and guided by the sealing element 118 on each side of the openings 126. In the embodiment shown in FIGS. 2 and 3 (specifically referred to the top view in FIG. 3), the lid portions 122 can be bottle-shaped, with a narrow guiding part 154 and a wider opening part 156. After opening the lid portions 122 by the mechanism described above, the test element 132, specifically the support body 136 of the test element 132, still may be in contact with the sealing element 118 in the region of the guiding part 154, which can allow for a radial lancing motion of the test element 132 with a well-defined direction, guided by the guiding part 154. The opening part 156 on the other hand can be wide enough to prevent the lancet 134 from touching the sealing element 118.

In FIG. 4, a second embodiment of a test element magazine 110 is depicted in a cross-sectional side view, similar to the view in FIG. 2. Again, in this embodiment, a test element 132 with a support body 136 can be comprised in a sealed chamber 114, e.g., facing radially outward in a disk-shaped test element magazine 110. Again, the test element magazine 110 can comprise a magazine body 116 and a sealing element 118. Each sealing element can again comprise a sealing support 120 and a number of lid portions 122, which may be shaped in a way similar to the embodiments shown in FIGS. 1 and 3. Other suitable shapes are possible.

Again, as in FIGS. 2 and 3, the chambers 114 can comprise a constricted portion 150. As opposed to the embodiment shown in FIGS. 2 and 3, this constricted portion 150 can comprise a number of ramp-shaped projections 158, which can be parts of the lid portions 122 and/or the magazine body 116. These ramp-shaped projections 158, protruding into the interior of the chamber 114, can be arranged close to the portion of the test elements 132 in which the lancet 134 meets the support body 136. As soon as the test element 132 is forced towards the left in FIG. 4 (performing a lancing motion pointing radially outwards), which may be driven by an actuator engaging the support body 136 of the test element 132 at the rear end in FIG. 4, the test element 132 can be lifted at its front end, thereby pushing open the lid portion 122 and thereby clearing the opening 126.

In FIG. 4, no membrane element 130 is depicted, but, in an alternative embodiment, an additional membrane element 130 may be provided for sealing the gaps between the lid portions 122 and the sealing support 120. The top view of the lid portions 122 can preferably be similar to the top view of the embodiment shown in FIG. 3, but other suitable embodiments may be used. The embodiment in FIG. 4 clearly shows that the design of the constricted portion 150 may vary, as the person skilled in the art will clearly notice, and still allow for an opening of the lid portions 122 without the active portions of the test elements 132, e.g., the lancets 134, touching any part of the scaling element 118 and/or the magazine body 116.

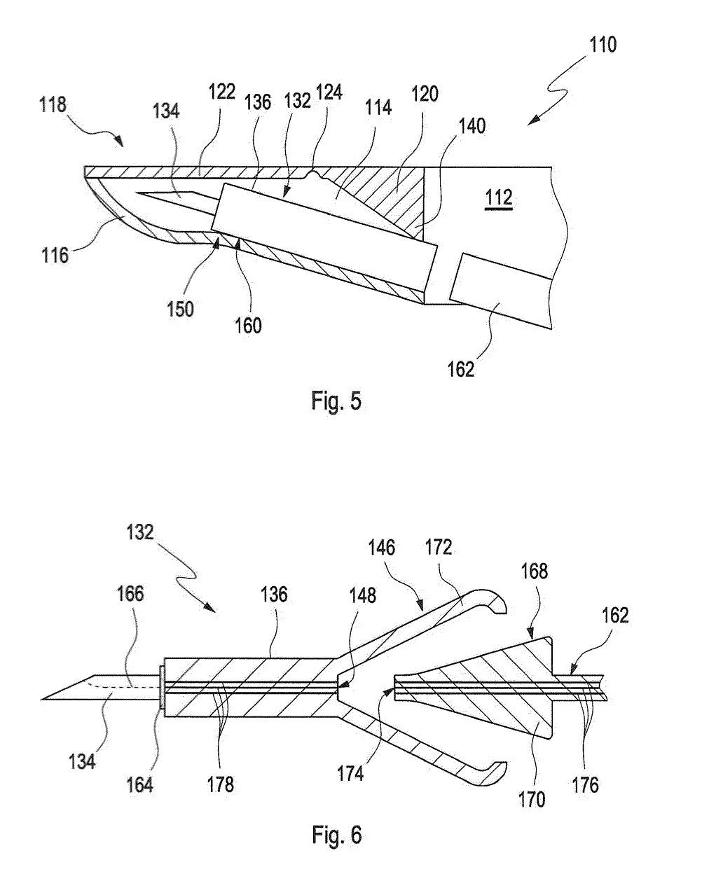

In FIG. 5, a fourth embodiment of a test element magazine 110 is depicted, in a similar view as shown in FIG. 4. In the embodiment shown in FIG. 5, which is more or less similar to the embodiment in FIG. 4, the constricted portion 150 can comprise a ramp 160 guiding the test element 132 at a sharp angle towards the lid portion 122 of the sealing element 118. The width of the support body 136 of the test element 132 and the arrangement of the ramp 160 can be designed in that the shoulder of the support body 136 can hit the lid portion 122 before the lancet 134 touches this lid portion 122. Thus, the lid portion 122 can be pushed open by swinging up the lid portion 122 around the hinge 124, without the lancet 134 touching the lid portion 122 or any other part of the magazine body 116 and/or the sealing element 118.

Further, similar to the embodiment shown in FIG. 2, the sealing support 120 of the sealing element 118 in the embodiment shown in FIG. 5 can comprise taper plugs 140 engaging the rear end of the support body 136 of the test element 134, thereby sealing the chamber 114 against a central opening 112 of the disk-shaped test element magazine 110. Further, in FIG. 5, it can be indicated schematically that the central opening 112 may be used for an actuator 162 to engage the test element 132 at its rear end, thereby mechanically forcing the test element 132 to perform a lancing motion. Further, the actuator 162 may be used for optically and/or electrically contacting the test element 132.

In FIG. 6, an exemplary embodiment of a test element 132 is shown in a cross-sectional side view. The test element 132 comprises an active portion, formed by a lancet 134 and a chemical substance 164. As described above, this chemical substance 164, which may e.g., be shaped like a substantially rectangular or round piece of film material at the transition between the lancet 134 and the support body 136 of the test element, may comprise one or more substances changing chemical and/or physical properties when getting into contact with a specific analyte. Thus, the chemical substance 164 may comprise one or more enzymes, such as gluconolactone, for detecting glucose in a body fluid sample, such as a blood sample.

For sampling, the lancet 134 can comprise a slit-formed capillary element 166, extending from the sharp edge of the lancet 134 towards the chemical substance 164. Thus, after perforating a skin portion of a living organism, such as a human or animal patient, a blood sample may be drawn by capillary forces through the capillary element 166 to the chemical substance 164. The capillary element 166 may comprise hydrophilic surfaces, in order to facilitate sampling of aqueous body fluids.

This support body 136 of the test element 132 may be used for mechanically fastening to the lancet 134. Further, the support body 136 may be used for mechanical engagement of the test element 132 by an engagement portion 168 of an actuator 162. This is schematically depicted in FIG. 6.

In the embodiment in FIG. 5, the actuator 162, for purposes of clarification only, is illustrated as a simple plunger. Nevertheless, the actuator 162, in the embodiment in FIG. 6, as well in the other embodiments, may comprise a more sophisticated engagement portion 168, allowing for several types of engagement between the actuator 162 and the test element 132. Thus, especially for the purpose of GAM actions, but also for test elements simply comprising a lancet 134, without any chemical substance 164, not only a forward, lancing motion may be desired, but also, after perforating a skin portion of a patient, a retraction movement. For this purpose, the engagement portion 168 of the actuator 162 may comprise a gripper engaging the rear-end engagement portion 146 of the test element 132. Additionally, or alternatively, as shown in the embodiment in FIG. 6, the actuator 162 and the engagement portion 168 of the actuator 162 may comprise a tapered plunger, comprising protrusions 170 engaging a clamp portion 172 of the engagement portion 146 of the test element 132. Thus, after pushing the front end of the plunger of the actuator 162 into the clamp portion 172 of the engagement portion 146 of the test element 132, a forward as well as a rearward movement of the test element 132 may be accomplished by an appropriate forward or retraction motion of the actuator 162.

Further, as also shown in the embodiment depicted in FIG. 6, besides a mechanical engagement, other types of engagement may be used. Thus, in the embodiment in FIG. 6, the engagement portion 168 of the actuator 162 can comprise an optical port 174, which can be connected to a number of optical fibers 176 inside the actuator 162. This optical port 174 may optically contact the optical port 148 inside the support body 136 of the test element 132. This optical port 148 of the test element 132 may be connected with the chemical substance 164 by a number of optical fibers 178 inside the support body 136. Thus, e.g., one optical fiber 178 may be provided (e.g., a central fiber) for guiding excitation light from a light source through the actuator 162 to the chemical substance 164, and two or more optical fibers 178 may be provided for guiding light emitted from the chemical substance 164 to an optical detector. Thus, optical measurements may be performed, in order to detect color changes of the chemical substance 164 and/or to detect changes in fluorescence behavior of the chemical substance 164, to determine the concentration of the analyte inside the body fluid sample. Other suitable optical detection methods are known and may be used alternatively, or additionally.

Other types of interconnection besides mechanical and optical interconnection may be used. Thus, the interconnection portions 168, 146 of the actuator 162 and the test elements 132 may be adapted to allow for an electrical interconnection of the test element 132. Thus, e.g., electrochemical measurements may be performed, such as electrochemical measurements using chemical substances 164 changing their electrochemical properties upon contact with the analyte.

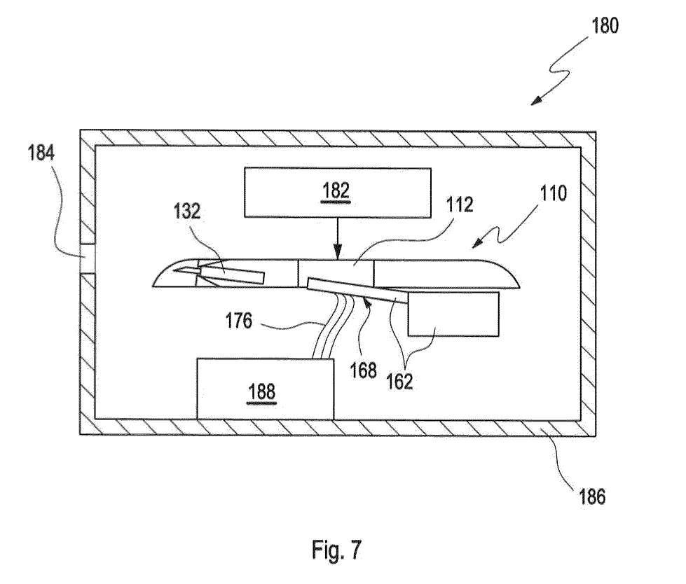

In FIG. 7, an embodiment of a test system 180 for measuring the concentration of at least one constituent (analyte) in a fluid sample is depicted in a simplified, schematic sectional side view. The test system 180 can comprises a test element magazine 110, such as the test element magazines 110 described above. Further, the test system 180 can comprise a rotating mechanism 182, which may be used for mechanically holding the test element magazine 110, as well as for positioning a specific test element chamber 114 in front of a lancing opening 184 within a housing 186 of the test system 180. The housing 186 may further comprise mechanical support elements for supporting some or all of the components of the test system 180.

The test system 180 can further comprise an actuator 162 for engaging one of the test elements 132 (only schematically shown in FIG. 7) in a measuring position inside the test element magazine 110.

The test system 180 may further comprise a measuring system 188. This measuring system 188 may provide an optical excitation source and one or more optical detectors, both connected via the optical fibers 176 to the engagement portion 168 of the actuator 162, for optically contacting the test element 132 and performing the measurement described above. The measuring system 188 may further comprise electronic components for performing the measurement described above, and may further comprise one or more computer elements, such as a microprocessor, as well as input/output means, storage means (e.g., a volatile and/or a non-volatile data storage) or other elements. The measuring system 188 may further be adapted for controlling the actuator 162, in order to control the overall sampling process.

It is noted that terms like “preferably,” “commonly,” and “typically” are not utilized herein to limit the scope of the claimed embodiments or to imply that certain features are critical, essential, or even important to the structure or function of the claimed embodiments. Rather, these terms are merely intended to highlight alternative or additional features that may or may not be utilized in a particular embodiment of the present disclosure.

For the purposes of describing and defining the present disclosure, it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the present disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

We claim:

1. A test element magazine, the test element magazine comprising at least one test element in at least one chamber, wherein:
the at least one chamber is individually sealed by a sealing element;
the at least one test element comprises at least one active portion for contacting a skin portion of a patient and/or for contacting a fluid sample; and
the at least one test element further comprises at least one support body, wherein the at least one support body is engaged by an actuator for effecting a lancing movement of the at least one test element or for performing a sampling motion, wherein by a forward movement of the at least one test element, the at least one support body of the at least one test element applies a force to the sealing element that forces open the sealing of the at least one chamber.

2. The test element magazine according to claim 1, wherein the at least one chamber comprises a constricted portion, the at least one chamber is dimensioned such that by forcing the at least one support body of the at least one test element into the constricted portion of the at least one chamber, a force is applied to the sealing element forcing open the sealing of the at least one chamber.

3. The test element magazine according to claim 2, wherein the constricted portion comprises at least one constriction element, the at least one construction element being a ramp, a cam protruding into the at least one chamber, a projection protruding into the at least one chamber, a shoulder protruding into the at least one chamber or combinations thereof.

4. The test element magazine according to claim 3, wherein the at least one constriction element is formed by a magazine body.

5. The test element magazine according to claim 4, wherein the magazine body is formed by a molded plastic element.

6. The test element magazine according to claim 1, wherein the sealing element comprises at least one lid portion that at least partially covers the at least one chamber.

7. The test element magazine according to claim 6, wherein the sealing element further comprises at least one sealing support, wherein the at least one lid portion is connected to the at least one sealing support by a hinge.

8. The test element magazine according to claim 7, further comprising a gap between the at least one sealing support and the at least one lid portion that is covered by a foil or membrane element.

9. The test element magazine according to claim 8, wherein the foil or membrane element is comprised of an aluminum foil, a copper foil, a polymer foil, a polyethylene foil, a polyethylene terephtalate foil, a fluorinated polyethylene foil, a metal-coated polymer foil, a polymer foil coated with an inorganic barrier material, a metal foil coated with silicon dioxide, or combinations thereof.

10. The test element magazine according to claim 6, wherein the at least one lid portion is a substantially rectangular shape, a lamellar shape, a shape of a piece of pie, or a bottle shape.

11. The test element magazine according to claim 6, further comprising an opening covered by the at least one lid portion that is narrower than the at least one test element width such that a perimeter of door openings restrains and guides the at least one test element when the at least one lid portion is in an open position.

12. The test element magazine according to claim 1, wherein the at least one chamber is dimensioned such that the at least one active portion of the at least one test element does not touch the sealing element during the process in which the body of the at least one test element forces open the sealing.

13. The test element magazine according to claim 1, wherein the test element magazine is a disk magazine.

14. The test element magazine according to claim 13, wherein a plurality of test elements are arranged radially in individual chambers, with the active portions of the test elements pointing outwards.

15. The test element magazine according to claim 13, wherein the disk magazine has an substantially circular shape.

16. The test element magazine according to claim 1, further comprises,
a disk body, wherein the sealing element at least partially covers the disk body.

17. The test element magazine according to claim 16, wherein the disk body comprises a molded plastic element.

18. The test element magazine according to claim 1, wherein the at least one active portion of the at least one test element comprises a lancet, an analytical test portion for detecting a constituent in a fluid sample, or combinations thereof.

19. The test element magazine according to claim 18, wherein the at least one test element comprises a capillary element for sampling a fluid sample.

20. The test element magazine according to claim 18, wherein the at least one test element comprises a coating.

21. The test element magazine according to claim 20, wherein the coating comprises a hydrophilic surface.

22. The test element magazine according to claim 1, wherein the at least one test element further comprises a substance that changes at least one chemical or physical property as a function of a concentration of the constituent in the fluid when bringing the fluid in contact with the substance.

23. The test element magazine according to claim 22, wherein the at least one chemical or physical property comprises an optical property.

24. The test element magazine according to claim 22, wherein the at least one chemical or physical property comprises an electrochemical property.

25. The test element magazine according to claim 1, wherein a part of the at least one support body of the at least one test element comprises a sealing portion, wherein the sealing portion forms part of the sealing of the at least one chamber.

26. The test element magazine according to claim 25, wherein a sterile part of the at least one test element is located inside the at least one chamber, and wherein an engagement portion of the at least one test element is located outside the at least one chamber.

27. A test system for measuring the concentration of at least one constituent in a fluid sample, the test system comprising:
a test element magazine according to claim 1; and
at least one actuator comprising an engagement portion for engaging a test element of the test element magazine.

28. The test system according to claim 27, wherein the actuator forces the support body of the test element into a constricted portion of the chamber forcing open the sealing of the chamber.

29. The test system according to claim 27, wherein the actuator forces apart at least part of the support body of the test element, thereby enlarging the width of the support body, thereby forcing open the sealing of the chamber.

30. The test system according to claim 27, wherein the engagement portion comprises an optical port for optically contacting the test element.

31. The test system according to claim 27, wherein the engagement portion comprises an electrical port for electrically contacting the test element.
